(19) United States
(12) Patent Application Publication
(10) Pub. No.: US 2019/0304587 A1
(43) Pub. Date: Oct. 3, 2019

(54) NUTRITION PERFORMANCE SCORECARD AND AUTOMATED SHOPPING LIST

(71) Applicant: Walmart Apollo, LLC, Bentonville, AR (US)

(72) Inventors: Robert Cantrell, Herndon, VA (US); Donald Ray High, Noel, MO (US)

(21) Appl. No.: 16/371,338

(22) Filed: Apr. 1, 2019

Related U.S. Application Data
(60) Provisional application No. 62/650,681, filed on Mar. 30, 2018.

(51) Int. Cl. G16H 20/60; G06Q 30/06; G06F 16/9032; G06F 16/906

(52) U.S. Cl. CPC G16H 20/60; G06Q 30/0631; G06F 16/906; G06Q 30/0641; G06F 16/90324; G06Q 30/0633

(57) ABSTRACT

A system, method and medium for creating an automated shopping and nutrition performance scorecard are discussed. A mobile computing device receives a list of grocery items and supplementary items selected by a user which are transmitted to a nutritional analysis module. The nutritional analysis module retrieves nutritional content information for each item in the list of grocery items and the supplementary items. The nutritional content information is aggregated based on categories. The aggregated categories are transmitted to the mobile computing device and displayed on a graphical user interface. An indication representative of a deviance from a recommended value is displayed for each aggregated category. The GUI provides the user with grocery item recommendations to lower excessive aggregated categories and raise deficient aggregated categories.

### RELATED APPLICATION

This application claims priority to, and the benefit of, U.S. Provisional Patent Application No. 62/650,681, entitled “Nutrition Performance Scorecard and Automated Shopping List”, filed Mar. 30, 2018, the contents of which ae incorporated herein by reference in their entirety.

### BACKGROUND

The process of grocery shopping for a consumer may attempt to consider the nutritional value of the groceries being acquired. Some nutritional information relating to nutritional categories is available on package labels. Other groceries, such as fruits and vegetables, may not have nutritional labeling.

### BRIEF DESCRIPTION OF DRAWINGS

Illustrative embodiments are shown by way of example in the accompanying drawings and should not be considered as a limitation of the present disclosure:

FIG. 1 is a block diagram illustrating a system for creating an automated shopping and nutrition performance scorecard according to an exemplary embodiment.

FIG. 2 is a diagram illustrating grocery shopping list on a mobile computing device according to an exemplary embodiment.

FIG. 3 is a diagram illustrating nutrition scorecard on a mobile computing device according to an exemplary embodiment.

FIGS. 4A and 4B are diagrams illustrating interactions with a nutrition scorecard and grocery shopping list according to an exemplary embodiment.

FIG. 4C is a diagram illustrating interactions with a grocery shopping list and supplementary items according to an exemplary embodiment.

FIG. 5 is a flow diagram illustrating a process for the creation and operation of a nutrition scorecard according to an exemplary embodiment.

FIG. 6 depicts a block diagram an exemplary computing device in accordance with an exemplary embodiment.

### DETAILED DESCRIPTION

Health conscious grocery shopping conventionally may be confusing, time consuming, and inconsistent Described in detail herein is a system to address health conscious grocery shopping by creating a nutrition performance scorecard and an automated shopping list. Embodiments of the system include a mobile computing device communicatively coupled to sensors and presenting a graphical user interface. The mobile computing device receives input of data relating to grocery items and supplementary items. The grocery items and supplementary items data is transmitted to a nutritional analysis module. The nutritional analysis module retrieves nutritional content information for the grocery items and the supplementary items, and aggregates nutritional category information based on the retrieved nutritional content information. The nutritional category information may be transmitted back to the mobile computing device to be displayed on a graphical user interface indicating threshold relationships between the nutritional categories and the nutritional content information of the grocery list items and the supplementary items.

In another embodiment, the graphical user interface may provide interactive objects corresponding to the nutritional categories that provide informational views of the grocery list based on the threshold relationships. In the case of excess values in nutritional categories, the graphical user interface may present a list of key grocery items accounting for disproportional amounts of the nutritional category information. Alternatively, in the case of deficiency, the graphical user interface may present a list of suggested grocery items to address the nutritional category deficiencies based on a shopping assistant module.

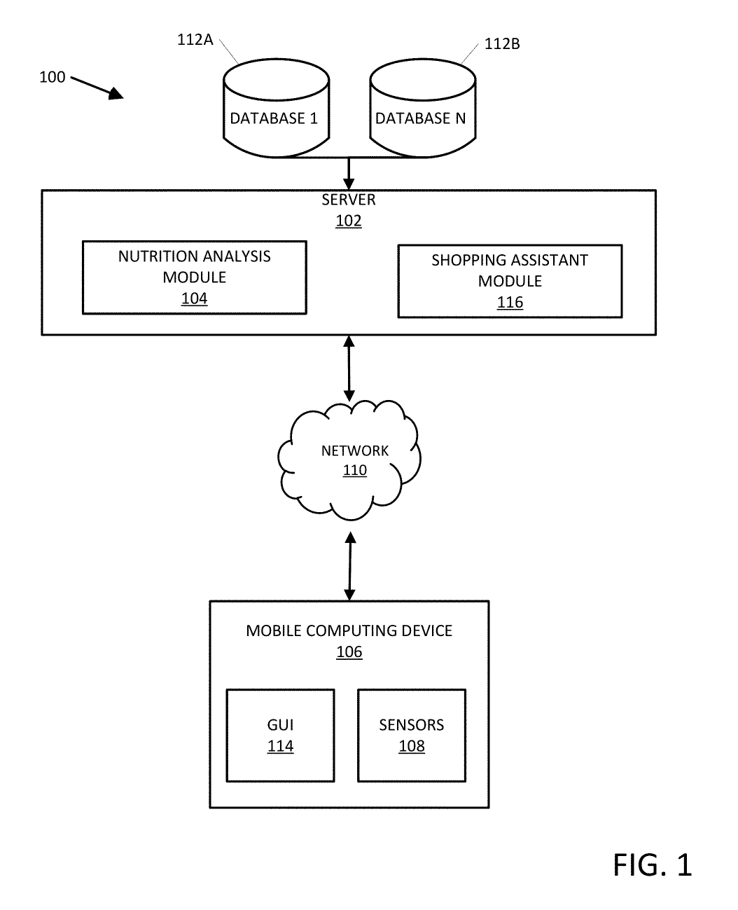

FIG. 1 is a block diagram illustrating a system for creating an automated shopping and nutrition performance scorecard according to an exemplary embodiment.

The system for creating an automated shopping and performance scorecard may include a server 102. The server 102 may be implemented as a standalone hardware processing device or an abstracted cloud-based processing device. Additionally, the server 102 may be virtualized within a physical hardware instance or executing natively on a hardware processing device.

A nutrition analysis module 104 may execute on the server 102. The nutrition analysis module 104 may be an application or service executing on the server 102 continuously. Alternately the nutrition analysis module 104 may periodically invoke through a system control daemon (e.g. systemd). The nutrition analysis module 104 may accept input from one or more mobile computing devices 106. The input for the nutrition analysis module 104 may include identifiers for products collected in a digital shopping basket or “cart.” The identifiers may correspond to but are not limited to a universal product codes to identify a specific product included in the “cart.” The nutritional analysis module 104 may also be configured to receive as input additional supplementary items, including restaurant prepared foods and other nutritional items not included within the “cart.” The nutrition analysis module 104 may provide output to one or more mobile computing devices 106. The output may include nutritional information corresponding to the items in the “cart” and other identified supplementary items. The nutritional information for an item or supplementary item may include a nutritional value as a measurement total (e.g. item contains 1 gram of x) or be provided with respect to a guideline (e.g. 30% of the recommended daily allowance of vitamin A). Additional output may include suggested alternative items that may be added to the grocery list in the “cart.” The nutritional analysis module 104 may provide the computational logic to retrieve nutritional information from the databases 112A, 112B corresponding to a grocery list or “cart,” compile the details of each nutritional category within each item in the grocery list, and intelligently recommend items to add or delete based on the compilation and a specified profile. The profile may be stored at the nutritional analysis module 104 after being selected by a user of the mobile computing device 106.

Additionally, a shopping assistant module 116 may execute like the nutrition analysis module 104 on the server 102. The shopping assistant module 116 may provide a list of suggested grocery items to the nutrition analysis module 104. The shopping assistant module 116 may evaluate a user's past purchases and identify common past purchases that may meet criteria set by the nutritional analysis module 104. The shopping assistant module 116 may interface with the databases 112A and 112B to retrieve and substitute items based on historical purchase patterns for the user.

Database 112A, 112B may be communicatively coupled to the server 102 and accessible by the nutrition analysis module 104. The databases 112A, 112B may provide an interface for the nutritional analysis module 106 to query information about a grocery item provided as input to the nutritional analysis module. The information in the database may be indexed or searchable based on a unique identifier correlating to the item or supplementary item. Indexed with the item or supplementary item may be nutritional content information for the item. The nutritional information may include nutritional information corresponding to one serving of the item, or alternatively nutritional information corresponding to the entire packaged contents of the item.

A network 110 communicatively couples the server 102 and the mobile computing device 106. The network 110 can be a wide area network (WAN) or the Internet. The network 110 can be operable to transport data packets compatible with mobile computing device 106 and the server 102. In one embodiment, compatible data packets can include data packets with transmission control protocol (TCP) or user datagram protocol (UDP) routing information, as well as an accessible application layer. The network 110 can interface with other networks or servers. The network 110 can be a combination of wired and wireless connection inclusively.

Mobile computing device 106 provides a front end into the system for producing a nutrition performance scorecard and shopping list. The mobile computing device 106, portrayed in this embodiment as a smart phone, may include any mobile computing device not limited to a smart phone, tablet, laptop PC, or wearable computing device. The mobile computing device 106 may include an operating system as well as an underlying networking stack allowing the utilization of the network. Additionally, the mobile computing device 106 may include a touchscreen display screen.

The mobile computing device 106 may include a graphical user interface (GUI) 114 with displayable items that respond when interfaced. The displayable items may include but are not limited to nutritional items and categories, grocery items, supplementary items, search boxes, and suggested items. The GUI 114 may display relevant information corresponding to the grocery items in a tabular format. Additionally the GUI 114 may display information in a visually distinct way including but not limited to color-coded variations intended to notify a user of nutritional deficiencies or excesses at a glance. For example, the GUI 114 may provide illustrative visualizations for caution thresholds corresponding to periodic allowances of nutrient values as well as illustrative visualizations for thresholds that exceed a periodic allowance threshold of particular types of nutrients. In the foregoing example the caution threshold may be lower than the periodic allowance threshold. The periodic allowance caution threshold and the periodic threshold may be based at least in part on referenced daily intake values from a dietary plan, a dietary restriction based on a health conditions, or a group of people (e.g. a family) and a body size of each of the group of people.

Attached or inclusive to the mobile computing device 106 may be an array of sensors 108. The sensors 108 may be utilized to input information about grocery items as well as supplementary items into the mobile computing device 106. Sensors 108 may include optical sensors such as barcode readers, quick response (QR) code readers, as well as optical imaging for item recognition software processing. Additionally, sensors 108 may include additional interfaces for reading machine readable tags including near field communication (NFC) readers. The sensors 108 may be integrated into the mobile computing device 106 or they may be communicatively coupled, either wired or wirelessly to the mobile computing device 106. It will be appreciated that the user may also type information regarding a grocery item or supplementary item in a user interface provided on the mobile computing device such as identifying a quantity of a type of fruit or vegetable for which no barcode or other similar identifier exists.

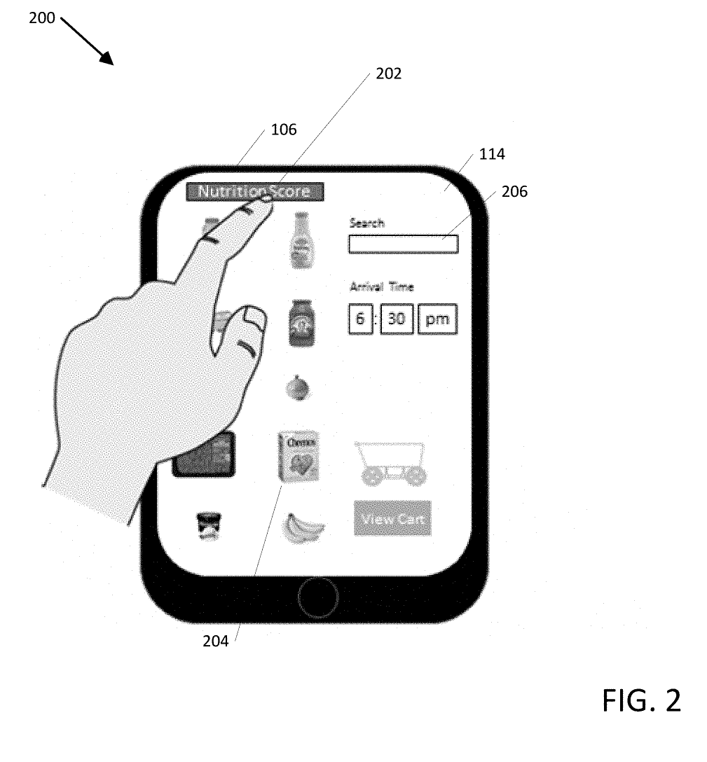

FIG. 2 is a diagram illustrating a grocery shopping list on a mobile computing device according to an exemplary embodiment.

The grocery shopping list may be rendered on the touchscreen display of the mobile computing device 106 in GUI 114. The GUI 114 may be implemented in platform specific development kits/stacks utilizing JAVASCRIPT, Objective C, or Universal Windows Platform interfaces. Alternatively, the GUI 114 may be implemented in platform agnostic development kits/stacks including Qt and SDL. The GUI 114 may include a nutrition score 202 selection button. Upon the selection of the nutrition score 202 selection button, the mobile computing device 106 may transmit the accumulated grocery list items from the mobile computing device 106 to the nutrition analysis module 104. A unique identifier corresponding to each grocery item 204 and unique to the packaged amount of the grocery item may be included in the transmission. A search box 206 may be included in the rendered GUI 114 for the addition of non-grocery list items.

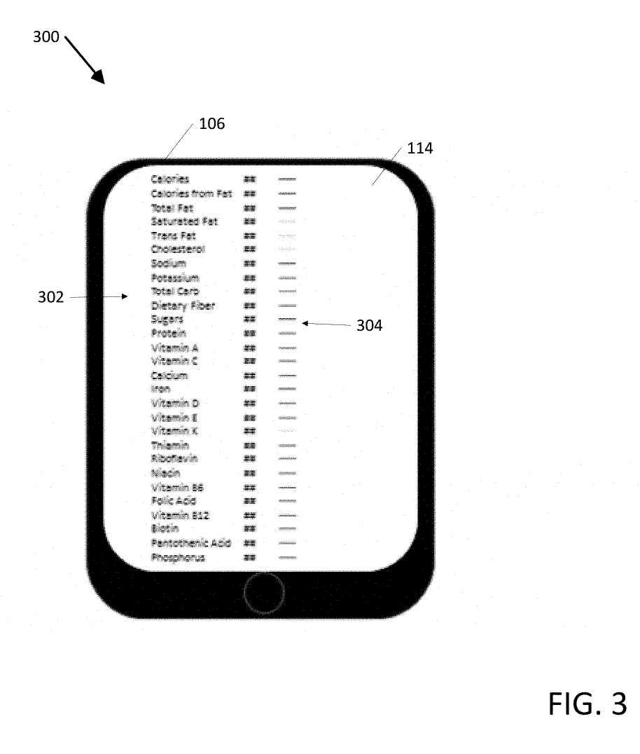

FIG. 3 is a diagram illustrating a nutrition scorecard 300 on a mobile computing device according to an exemplary embodiment. The mobile computing device 106 may render a GUI 114 on the display. The nutrition scorecard 300 may be updated based on the selection of the nutrition score 202 selection button. The nutrition scorecard 300 may include aggregated nutrition categories 302 based on the grocery items 204 in the “cart” prior to the selection of the nutrition scorecard 300. After the transmission of the unique identifiers to the nutritional analysis module 104 from the mobile computing device 106, the nutritional analysis module transmits the aggregated nutrition categories 302 to the GUI 114. Additionally the nutritional analysis module transmits aggregated nutritional information 304. The GUI 114 may interpret and display the aggregated nutritional information 304 in many different ways. In one embodiment, the aggregated nutritional information 304 may be illustrated in a color-coded manner where an indication red may be an indication of excess or deficiency based on scale of recommended nutrient ingestion guidelines. Alternatively the aggregated nutritional information 304 may be illustrated where green is inside a deviation around an optimal value based on a scale of recommended nutrient ingestion guidelines. An illustration of orange may indicate a value outside a deviation based on the scale that is however not within the range of deficiency or excess.

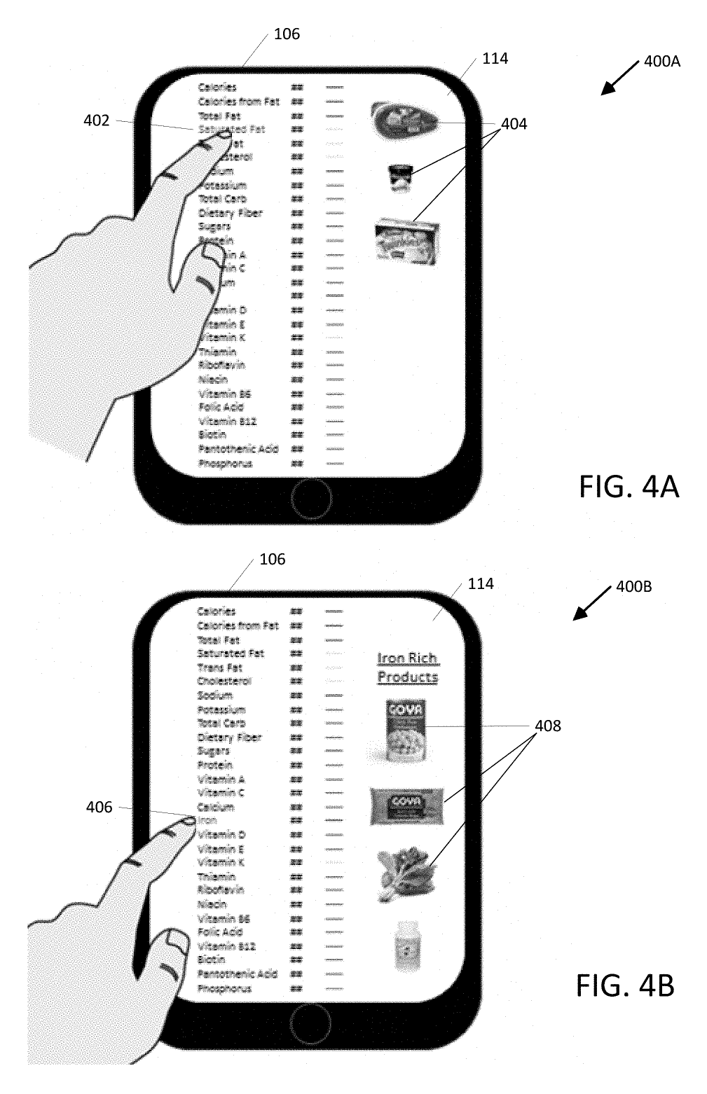

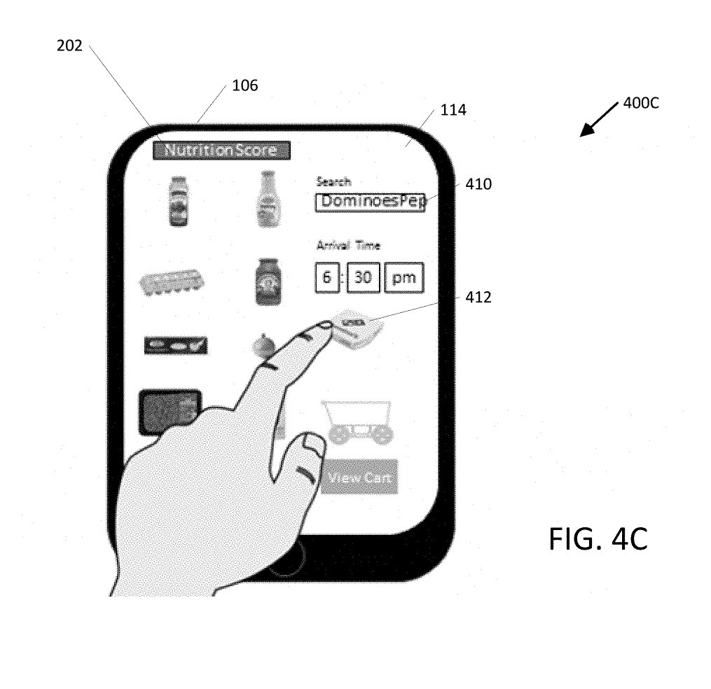

FIGS. 4A, 4B, and 4C are diagrams 400A, 400B illustrating interactions with a nutrition scorecard and grocery shopping list according to an exemplary embodiment.

In FIG. 4A, a user's selects one of the aggregated nutrition categories 302. The selected nutrition category 402 provides a break out of the grocery items contributing to the aggregated nutritional information 304. In this example, three grocery items 404 are identified as items in the grocery list contributing to the aggregated nutritional information 304. The three grocery items 404 may be selected for display in the GUI 114 by the percentage amount that each of the items contributes excessive aggregated nutritional information 304. Alternatively, the three items grocery items 404 may be determined based on their impact to other deficient or excess categories as well. The GUI 114 is not limited to only displaying three items as this is just a non-limiting exemplarily embodiment any implementation is only limited by the mobile computing device's displaying mechanism and the implementation of the GUI 114.

Similarly to FIG. 4B, a selected nutrition category 406 may provide suggestions, provided by the shopping assistant module 116, in order to resolve an excessive or deficient aggregate nutrient category. In this non-limiting example, the user selects the selected nutrition category 406. In this example, the selected nutritional category 406 is deficient in aggregate nutritional information. The GUI 114 may suggest alternative items to supplement other items in the grocery list to bring the nutrient category closer to the recommended value. Suggested shopping information 408 may be displayed in the GUI 114 to allow the user to select an item, thereby adding it to the grocery list. The suggested shopping information 408 from the shopping assistant module 116 may utilize information obtained from the database 112A, 112B to determine past purchased items that meet the nutritional requirement to move the aggregate nutritional category closer to a recommended range. Alternatively, the suggested shopping information 408 may utilize information in the databases 112A, 112B to determine grocery items that have the most impact in the affected category and the least impact in other unaffected nutritional categories.

FIG. 4C is a diagram 400C illustrating interactions with a grocery shopping list and supplementary items according to an exemplary embodiment. The GUI 114 provides the user with a search box 410 for the entering of supplementary items that may impact nutritional aggregate information. The search box 410 may be implemented as a text box. Upon the user interfacing with the search box 410, any text entered in the search box may be transmitted to the nutritional analysis module 104. The nutritional analysis module 104 may access the database to determine a best match item based on the text transmitted from the GUI 114. The nutritional analysis module 104 transmits an indication of potential supplementary items to be included in the nutrition scorecard 202. The user may select an accurate representation of the supplementary item 412 to include any nutritional information from the supplementary item into the nutritional score card. Upon the interaction with the nutritional score 202 selection button, the mobile computing device 106 transmits the unique identifiers of the grocery items with a unique identifier corresponding to the supplementary items to generate the corresponding aggregate nutrient categories 302. Alternatively, the user may utilize sensors 108 communicatively coupled to the mobile computing device 106 to input a supplementary item 412. The user may use a sensor such as a barcode scanner to scan a product's UPC or QR code. Additionally, the user may use an optical imaging device to photograph or image a food item to be image processed and identified for representative nutritional information. Upon the selection of the nutritional scorecard 202 selection button, unique identifiers corresponding to the sensor-scanned items may be transmitted to the nutritional analysis module 104.

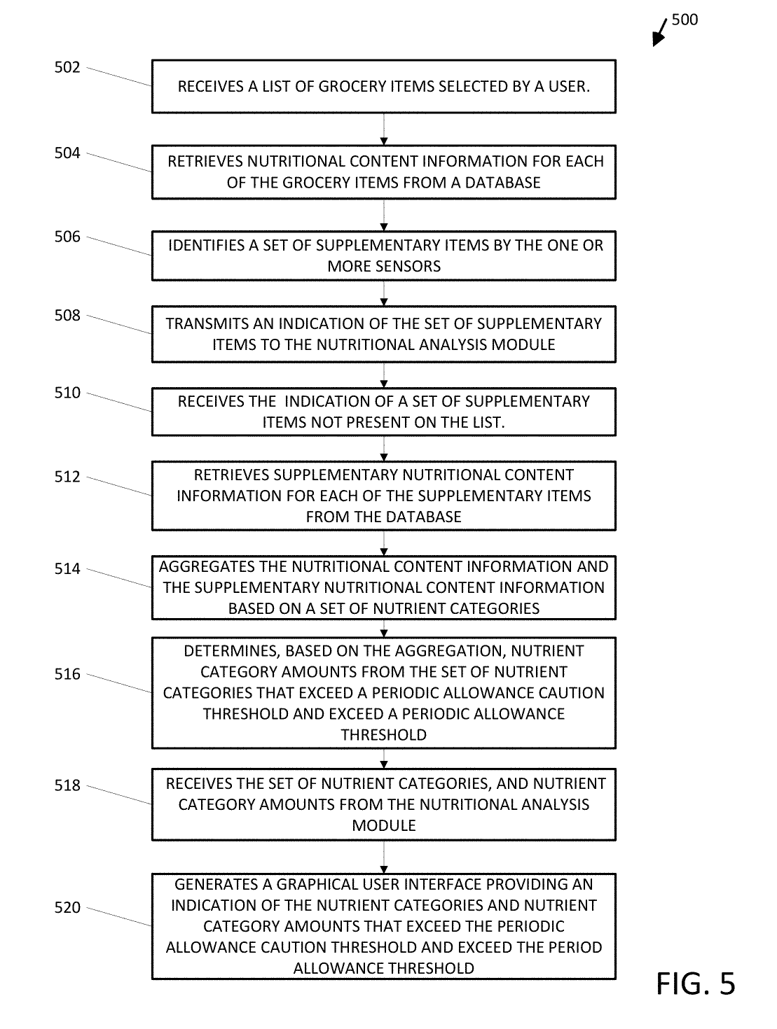

FIG. 5 is a flow diagram illustrating a process for the creation and operation of a nutrition scorecard according to an exemplary embodiment.

At step 502, the nutritional analysis module receives a list of grocery items selected by a user, from a mobile computing device. The items may be input by a GUI 114 and scanned by sensors 118. A user may utilize the mobile computing device as a shopping tool for the identification of grocery items the user wishes to purchase.

At step 504, the nutritional analysis module, retrieves nutritional content information of each of the grocery items from a database. The nutritional content information may correspond to details provided on the nutrition fact labeling. However the content information may not be limited to nutrition facts and may be obtained through independent testing, crowd sourcing or from the producer of the item. The nutritional content information may be stored in the databases 112A, 112B.

At step 506, the mobile computing device identifies a set of supplementary items by the one or more sensors. Supplementary items may be imported into the grocery list by the user utilizing manual input with a search box, or sensors configured to scan and identify various other supplementary items not on the grocery list.

At step 508, the mobile computing device transmits an indication of the set of supplementary items to the nutritional analysis module. The supplementary items may be encoded by the mobile computing device and transmitted to the nutritional analysis module utilizing a networking protocol that the nutritional analysis module may accept and decode.

At step 510, the nutritional analysis module receives the indication of a set of supplementary items not present on the list. The nutritional analysis module decodes the supplementary items and utilizes unique identifiers to include the supplementary items in the grocery list.

At step 512, the nutritional analysis module retrieves supplementary nutritional content information for each of the supplementary items from the database. The nutritional analysis module queries the databases 112A, 112B for nutritional content information pertaining to the list of supplementary items. The nutritional content information for the supplementary items may be provided by the producer of the supplementary items, crowdsource nutritional content values, and image analysis of photographic images of each of the supplementary items. Alternatively, the nutritional analysis module may query databases under control of third parties responsible for providing nutritional content analysis for the supplementary items.

At step 514, the nutritional analysis module aggregates the nutritional content information and the supplementary nutritional content information based on a set of nutrient categories that are applicable to the list of items in the list of groceries.

At step 516, the nutritional analysis module determines, based on the aggregation, nutrient category amounts from the set of nutrient categories that exceed a periodic allowance caution threshold and exceed a periodic allowance threshold, wherein the periodic allowance caution threshold is lower than the periodic allowance threshold. Periodic allowances may be determined based on governmental administrative entities (e.g. U.S.D.A.) guidelines or may be individually selected by a user based on dietary desires. For example, seasonal diets may be utilized to determine periodic allowances as well as “fad diet” plans.

At step 518, the mobile computing device receives the set of nutrient categories, and nutrient category amounts from the nutritional analysis module.

At step 520, the mobile computing device generates a graphical user interface providing an indication of the nutrient categories and nutrient category amounts that exceed the periodic allowance caution threshold and exceed the period allowance threshold.

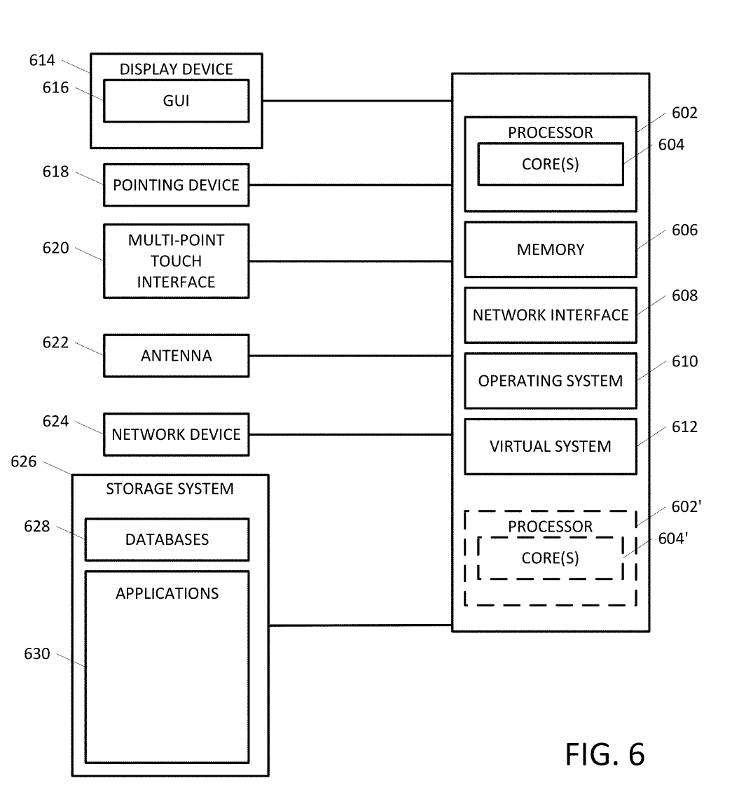

FIG. 6 is a block diagram of an example computing device for implementing exemplary embodiments of the present disclosure. Computing device 600 can implement embodiments for creating an automated shopping and nutrition performance scorecard. For example, the computing device can be embodied as a portion of the server, sensors, and mobile computing device. The computing device 600 includes one or more non-transitory computer-readable media for storing one or more computer-executable instructions or software for implementing exemplary embodiments. The non-transitory computer-readable media may include, but are not limited to, one or more types of hardware memory, non-transitory tangible media (for example, one or more magnetic storage disks, one or more optical disks, one or more flash drives, one or more solid state disks), and the like. For example, memory 606 included in the computing device 600 may store computer-readable and computer-executable instructions or software for implementing exemplary operations of the computing device 600. The computing device 600 also includes configurable and/or programmable processor 602 and associated core(s) 604, and optionally, one or more additional configurable and/or programmable processor(s) 602′ and associated core(s) 604′ (for example, in the case of computer systems having multiple processors/cores), for executing computer-readable and computer-executable instructions or software stored in the memory 606 and other programs for implementing exemplary embodiments of the present disclosure. Processor 602 and processor(s) 602′ may each be a single core processor or multiple core (604 and 604′) processor. Either or both of processor 602 and processor(s) 602′ may be configured to execute one or more of the instructions described in connection with computing device 600.

Virtualization may be employed in the computing device 600 so that infrastructure and resources in the computing device 600 may be shared dynamically. A virtual machine 612 may be provided to handle a process running on multiple processors so that the process appears to be using only one computing resource rather than multiple computing resources. Multiple virtual machines may also be used with one processor.

Memory 606 may include a computer system memory or random access memory, such as DRAM, SRAM, EDO RAM, and the like. Memory 606 may include other types of memory as well, or combinations thereof. The computing device 600 can receive data from input/output devices. A user may interact with the computing device 600 through a visual display device 614, such as a computer monitor, which may display one or more graphical user interfaces 616, multi touch interface 620 and a pointing device 618.

The computing device 600 may also include one or more storage devices 626, such as a hard-drive, CD-ROM, or other computer readable media, for storing data and computer-readable instructions and/or software that implement exemplary embodiments of the present disclosure. For example, exemplary storage device 626 can include one or more databases 628 for storing information associated sensor reading and data related to product shelf life. The databases 628 may be updated manually or automatically at any suitable time to add, delete, and/or update one or more data items in the databases.

The computing device 600 can include a network interface 608 configured to interface via one or more network devices 624 with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, controller area network (CAN), or some combination of any or all of the above. In exemplary embodiments, the computing system can include one or more antennas 622 to facilitate wireless communication (e.g., via the network interface) between the computing device 600 and a network and/or between the computing device 600 and other computing devices. The network interface 608 may include a built-in network adapter, network interface card, PCMCIA network card, card bus network adapter, wireless network adapter, USB network adapter, modem or any other device suitable for interfacing the computing device 600 to any type of network capable of communication and performing the operations described herein.

The computing device 600 may run any operating system 610, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, or any other operating system capable of running on the computing device 600 and performing the operations described herein. In exemplary embodiments, the operating system 610 may be run in native mode or emulated mode. In an exemplary embodiment, the operating system 610 may be run on one or more cloud machine instances.

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes multiple system elements, device components or method steps, those elements, components, or steps can be replaced with a single element, component, or step. Likewise, a single element, component, or step can be replaced with multiple elements, components, or steps that serve the same purpose. Moreover, while exemplary embodiments have been shown and described with references to particular embodiments thereof, those of ordinary skill in the art will understand that various substitutions and alterations in form and detail can be made therein without departing from the scope of the present disclosure. Further, still, other aspects, functions, and advantages are also within the scope of the present disclosure.

Exemplary flowcharts are provided herein for illustrative purposes and are non-limiting examples of methods. One of ordinary skill in the art will recognize that exemplary methods can include more or fewer steps than those illustrated in the exemplary flowcharts and that the steps in the exemplary flowcharts can be performed in a different order than the order shown in the illustrative flowcharts.

We claim:

1. A system for dynamically creating a nutritional scorecard based on a shopping list comprising:
a database holding nutritional content information;
a server communicatively coupled to the database and configured to execute a nutritional analysis module that when executed:
receives a list of grocery items selected by a user,
retrieves a nutritional content information of each of the grocery items from the database,
receives an indication of a set of supplementary items not present on the list,
retrieves supplementary nutritional content information for each of the supplementary items from the database,
aggregates the nutritional content information and the supplementary nutritional content information based on a set of nutrient categories applicable to the items in the list of groceries, and
determines, based on the aggregation, nutrient category amounts from the set of nutrient categories that exceed a periodic allowance caution threshold and exceed a periodic allowance threshold, wherein the periodic allowance caution threshold is lower than the periodic allowance threshold, and
a mobile computing device, comprising one or more sensors, communicatively coupled to the server and configured to execute a suggestion shopping module that when executed:
identifies the set of supplementary items by the one or more sensors,
transmits an indication of the set of supplementary items to the nutritional analysis module;
receives the set of nutrient categories, and nutrient category amounts from the nutritional analysis module,
generates a graphical user interface providing an indication of the nutrient categories and nutrient category amounts that exceed the periodic allowance caution threshold and exceed the periodic allowance threshold.

2. The system of claim 1, wherein the nutritional analysis module when executed further:
identifies a grocery item, from the list of grocery items, with a nutrient category amount exceeding a proportional threshold, wherein the proportional threshold correlates to a proportion of the nutrient category amount,
identifies a set of substitute grocery items for the grocery item based on a lowering of the nutrient category amount, wherein the items in the set of substitute grocery items lower the nutrient category amount,
sends the set of substitute grocery items to the shopping assistant module,
receives a selection of the set of substitute grocery items from the shopping assistant module,
retrieves nutritional content information for the selection from the database,
updates the nutrition category amounts, based on the nutritional content information of the selection,
updates the indication of each of the set of nutrient categories based on the updated nutrition category amounts wherein the nutrition category amounts exceed the periodic allowance caution threshold or exceed the periodic allowance threshold, and
send the updated indications to the mobile computing device for display on the graphical user interface.

3. The system of claim 2, wherein the set of substitute grocery items are based on historical purchase patterns.

4. The system of claim 1 wherein the periodic allowance caution threshold and periodic allowance threshold are based at least in part on reference daily intake values.

5. The system of claim 1 wherein the periodic allowance caution threshold and periodic allowance threshold are based at least in part on a dietary restriction based on health conditions.

6. The system of claim 1 wherein the periodic allowance caution threshold and periodic allowance threshold are based at least in part on a plurality of people and a body size of each of the plurality of people.

7. The system of claim 1, the one or more sensors comprising an imaging device configured to read universal product codes (UPC), quick response (QR) codes, and direct image capture.

8. A method for dynamically creating a nutritional scorecard based on a shopping list comprising:
receiving, from a mobile computing device, a list of grocery items selected by a user,
retrieving a nutritional content information of each of the grocery items from a database,
identifying the set of supplementary items by the one or more sensors,
transmitting an indication of the set of supplementary items to the nutritional analysis module;
receiving, by the nutritional analysis module, the indication of a set of supplementary items not present on the list,
retrieving supplementary nutritional content information for each of the supplementary items from the database,
aggregating the nutritional content information and the supplementary nutritional content information based on a set of nutrient categories, applicable to the list of items in the list of groceries,
determining, based on the aggregation, nutrient category amounts from the set of nutrient categories that exceed a periodic allowance caution threshold and exceed a periodic allowance threshold, wherein the periodic allowance caution threshold is lower than the periodic allowance threshold,
receiving, by a mobile computing device, comprising one or more sensors, the set of nutrient categories, and nutrient category amounts from the nutritional analysis module, and
generating a graphical user interface providing an indication of the nutrient categories and nutrient category amounts that exceed the periodic allowance caution threshold and exceed the period allowance threshold.

9. The method of claim 8, further comprising:
identifying a grocery item, from the list of grocery items, with a nutrient category amount exceeding a proportional threshold, wherein the proportional threshold correlates to a proportion of the nutrient category amount,
identifying a set of substitute grocery items for the grocery item based on a lowering of the nutrient category amount, wherein the items in the set of substituted grocery items lower the nutrient category amount,
sending the set of substitute grocery items to a shopping assistant module,
receiving a selection of the set of substitute grocery items from the shopping assistant module,
retrieving nutritional content information for the selection from the database;
updating the nutrition category amounts, based on the nutritional content information of the selection;
updating the indication of each of the set of nutrient categories based on the updated nutrition category amounts wherein the nutrition category amounts exceed the periodic allowance caution threshold or exceed the periodic allowance threshold; and
sending the updated indications to the mobile computing device for display on a graphical user interface.

10. The method of claim 9, wherein the set of substitute grocery items are based on historical purchase patterns.

11. The method of claim 8 wherein the periodic allowance caution threshold and periodic allowance threshold are based at least in part on reference daily intake values.

12. The method of claim 8 wherein the periodic allowance caution threshold and periodic allowance threshold are based at least in part on a dietary restriction based on health conditions.

13. The method of claim 8 wherein the periodic allowance caution threshold and periodic allowance threshold are based at least in part on a plurality of people and a body size of each of the plurality of people.

14. The method of claim 8, the one or more sensors, comprising an imaging device configured to read universal product codes (UPC), quick response (QR) codes, and perform direct image capture.

15. A non-transitory computer readable medium for dynamically creating a nutritional scorecard based on a shopping list, having stored thereon, instructions that when executed in a computing system, cause the computing system to perform operations comprising:
receiving, from a mobile computing device, a list of grocery items selected by a user,
retrieving a nutritional content information of each of the grocery items from a database,
identifying the set of supplementary items by the one or more sensors,
transmitting an indication of the set of supplementary items to the nutritional analysis module;
receiving, by the nutritional analysis module, the indication of a set of supplementary items not present on the list,
retrieving supplementary nutritional content information for each of the supplementary items from the database,
aggregating the nutritional content information and the supplementary nutritional content information based on a set of nutrient categories, applicable to the list of items in the list of groceries,
determining, based on the aggregation, nutrient category amounts from the set of nutrient categories that exceed a periodic allowance caution threshold and exceed a periodic allowance threshold, wherein the periodic allowance caution threshold is lower than the periodic allowance threshold,
receiving, by a mobile computing device, comprising one or more sensors, the set of nutrient categories, and nutrient category amounts from the nutritional analysis module, and
generating a graphical user interface providing an indication of the nutrient categories and nutrient category amounts that exceed the periodic allowance caution threshold and exceed the period allowance threshold.

16. The computer readable medium of claim 15, the instructions further comprising:
identifying a grocery item, from the list of grocery items, with a nutrient category amount exceeding a proportional threshold, wherein the proportional threshold correlates to a proportion of the nutrient category amount,
identifying a set of substitute grocery items for the grocery item based on a lowering of the nutrient category amount, wherein the items in the set of substituted grocery items lower the nutrient category amount,
sending the set of substitute grocery items to a shopping assistant module,
receiving a selection of the set of substitute grocery items from the shopping assistant module,
retrieving nutritional content information for the selection from the database,
updating the nutrition category amounts, based on the nutritional content information of the selection,
updating the indication of each of the set of nutrient categories based on the updated nutrition category amounts wherein the nutrition category amounts exceed the periodic allowance caution threshold or the periodic allowance threshold, and
sending the updated indications to the mobile computing device for display on a graphical user interface.

17. The computer readable medium of claim 16, wherein the set of substitute grocery items are based on historical purchase patterns.

18. The computer readable medium of claim 15 wherein the periodic allowance caution threshold and periodic allowance threshold are based at least in part on reference daily intake values.

19. The computer readable medium of claim 15 wherein the periodic allowance caution threshold and periodic allowance threshold are based at least in part on a dietary restriction based on health conditions.

20. The computer readable medium of claim 15, the one or more sensors comprising an imaging device configured to read universal product codes (UPC), quick response (QR) codes, and direct image capture.
